SEQUENCE LISTING (3 sequences)

SEQ ID NO: 1 - protein, 6 aa, Artificial
  synthetic peptide
VGVAPG

SEQ ID NO: 2 - protein, 5 aa, Artificial
  synthetic peptide
KIGRK

SEQ ID NO: 3 - protein, 4 aa, Artificial
  synthetic peptide
LIGK

(19) United States
(12) Patent Application Publication
(10) Pub. No.: US 2008/0268077 A1
(43) Pub. Date: Oct. 30, 2008

(54) Process for Strengthening the Barrier Function of Undamaged Skin

(75) Inventor: Gabriele Vielhaber, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/576,937

(22) PCT Filed: Oct. 14, 2005
(86) PCT No.: PCT/EP2005/055254

§ 371 Date: Apr. 9, 2007

Related U.S. Application Data
(60) Provisional application No. 60/618,840, filed on Oct. 14, 2004.

(51) Int. Cl. A61K 31/164; A61K 31/045; A61K 36/906; A61K 31/221; A61P 17/00; A61P 37/08

(52) U.S. Cl. 424/756; 514/729; 514/625; 514/551

(57) ABSTRACT

A process is described for strengthening the barrier function of undamaged skin in particular against allergens and/or for preventing or inhibiting an allergic reaction of undamaged skin on contact with an allergenic active ingredient,

with the following step:
Preparation of a mixture comprising:
(c) a ceramide and/or a pseudoceramide and
(d) an anti-irritant
Application of an effective amount of the mixture to the undamaged skin.

The present invention concerns processes for strengthening the barrier function of undamaged skin and corresponding processes for the cosmetic treatment of undamaged skin with a cosmetic active ingredient having an allergenic side-effect. Within the context of the invention, mixtures are used which contain (a) a ceramide and/or a pseudoceramide and (b) an anti-irritant. There is a particular emphasis on cosmetic and dermatological applications for the preventive protection of undamaged skin against contact allergies. The term “skin” here also includes the oral mucosa.

The skin is the largest bodily organ. It protects the body against uncontrolled water loss and environmentally induced mechanical, physical, biological and chemical stress. This protective function is primarily fulfilled by the so-called epidermal permeability barrier. This is located in the topmost layer of skin, the stratum corneum, and consists of a compact network of multiple intercellular lipid lamellae and horny cells embedded within them.

The gums and oral mucosa also have a permeability barrier, the essential constituents of which are lipid lamellae [Law et al. (1995) Arch. Oral Biol. 40: 1085-1091].

Numerous investigations have verified that the intercellular lipid lamellae are composed of cholesterol, ceramides and fatty acids in a molar ratio of 1:1:1. Of these, the lipid class of ceramides (N-acyl sphingosines) is of particular importance, firstly since ceramides make up almost 50% of the proportion by weight of barrier lipids. Secondly, special ceramides carrying a long-chain ω-hydroxy fatty acid (C30-32) allow a covalent bonding to glutamate radicals of surface proteins in the horny cells. This ensures that the permeability barrier has a particularly rigid structure.

Many factors are known to damage the barrier of skin (scalp) and hair, including excessive treatment with detergents or solvents, irradiation with UV light or inadequate air humidity. In addition, however, many eczematous disease profiles such as e.g. atopic and seborrhoeic dermatitis and dandruff, or serious hereditary diseases such as Gaucher disease are characterised by a barrier disorder. Finally, a deterioration in the barrier function of the skin is observed with advancing years.

If the barrier function is reduced, topically applied substances such as e.g. allergens and irritants can penetrate into the skin more easily, where more severe effects can develop than in undamaged skin. Attempts have therefore already been made to strengthen the barrier function of the skin to prevent allergic and irritative skin reactions. Corresponding active ingredients have been described in DE 4420625 and DE 3330628 (bisabolol or/and panthenol), WO 02069911, DE 10111045, WO 9712598, WO 01021150 (ceramide biosynthesis stimulators) and FR 2811228 (oligosaccharides in combination with ceramide 2).

Another possibility for preventing the cited skin damage or for repairing it, is the topical application of ceramide-containing dermatological compositions [Kucharekova et al. (2002) Contact Dermatitis 46: 331-338; Coderch et al. (2002) Contact Dermatitis 47: 139-146].

A preventive action against skin damage is preferable to a repair action, since the biological reactions triggered by the occurrence of skin damage propagate within a cell and into the deeper layers of the skin in a cascade effect. Hitherto it has not been possible to stop these secondary reactions completely or even reverse them by treatment with active ingredients. In the case of prevention, the damage does not occur at all, so secondary reactions are also avoided.

Natural ceramides are difficult to isolate. Their synthesis is also complicated and expensive. In addition, the processing of natural ceramides in emulsions presents difficulties because of their high melting point. For that reason structural analogues of ceramides (pseudoceramides) have been developed.

U.S. 06060612 describes the synthesis of 1,3-bis-(N-(2-hydroxyethyl)alkylamino)-2-hydroxypropanes as pseudoceramides and their cosmetic use.

WO 9821176 describes the production of N-(2-hydroxyethyl)-3-oxo-2-alkyl alkylamides and their cosmetic use for protection against skin ageing and to strengthen the resistance of and to repair skin and hair.

EP 0864563 A1 describes the use of N-acyl hydroxyamino acid esters, in particular N-acyl hydroxyproline and threonine esters, to strengthen the natural barrier function to protect against external influences and irritations.

The synergistic combination of N-acyl hydroxyamino acid esters with bisabolol for skin and hair repair is described in Research Disclosure No. 468117 (May 2003). The preventive use of this combination is not mentioned, however.

The object of the present invention was to provide a process for strengthening the barrier function of undamaged (in particular unirritated) skin (including the oral mucosa) against allergens in particular and/or for preventing or inhibiting an allergic reaction of undamaged skin on contact with an allergenic active ingredient. Accordingly, a further object of the present invention was to provide a process for the cosmetic treatment of undamaged skin with a cosmetic active ingredient having (usually) an allergenic side-effect, wherein there is little or no allergic reaction in the skin. The preparations and individual active ingredients to be used in the process to be provided according to the invention should be inexpensive and effective. The barrier of the undamaged skin should be strengthened as a preventive measure and contact allergies either prevented or at least inhibited (reduced) more effectively than is possible in accordance with the prior art.

The stated object(s) is (are) achieved according to the invention by a process for strengthening the barrier function of undamaged (in particular unirritated) skin against allergens in particular and/or for preventing or inhibiting an allergic reaction of undamaged skin on contact with an allergenic active ingredient,

with the following step:
Preparation of a mixture comprising:
(a) a ceramide and/or a pseudoceramide and
(b) an anti-irritant
Application of an effective amount of the mixture to the undamaged skin.

Corresponding to this is a process according to the invention for the cosmetic treatment of undamaged (in particular unirritated) skin with a cosmetic active ingredient having an allergenic side-effect, with the following steps:
Preparation of a mixture comprising:
(a) a ceramide and/or a pseudoceramide and
(b) an anti-irritant and optionally
(c) an acceptable support
Application of an effective amount of the mixture to the undamaged skin so that its barrier function is strengthened and at the same time or thereafter
Application of the cosmetic active ingredient having an allergenic side-effect to the skin.

In addition to the stated process, the present invention also provides the use of a mixture comprising

(a) a ceramide and/or a pseudoceramide and
(b) an anti-irritant
to produce a composition to strengthen the barrier function of undamaged skin and/or to prevent allergic skin reactions, in particular contact allergies.

The terms “ceramide”, “pseudoceramide” and “anti-irritant” all cover individual compounds and mixtures of two or more ceramides, pseudoceramides or anti-irritants.

The invention is based on the surprising discovery that the topical application of a mixture containing (a) a ceramide and/or a pseudoceramide and (b) an anti-irritant can effectively prevent allergic skin reactions caused by some fragrances, for example. The mixtures for use according to the invention surprisingly strengthen the barrier function of undamaged skin synergistically and thus prevent or inhibit the allergic reaction of the undamaged skin on contact with an allergenic active ingredient. The processes and uses according to the invention are ideally suitable in particular for the prevention of fragrance-induced contact allergies. Within the context of the present invention the use of pseudoceramides is preferred for economic reasons because of the poor availability of natural ceramides as mentioned above.

Anti-irritants within the meaning of the present invention are anti-inflammatory active ingredients or active ingredients to relieve reddening and itching that are suitable for or commonly used for dermatological applications. Substances which reduce the amount of cytokines, interleukins, prostaglandins and/or leukotrienes in cells and tissue are preferred.

Steroidal anti-inflammatory substances of the corticosteroid type, such as e.g. hydrocortisone, dexamethasone, dexamethasone phosphate, methyl prednisolone or cortisone, are advantageously used as anti-inflammatory active ingredients or active ingredients relieving reddening and itching (anti-irritants), the list of which can be extended by the addition of other steroidal anti-inflammatories. Non-steroidal anti-inflammatories can also be used. Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Alternatively, natural anti-inflammatory substances or substances to relieve reddening and itching can be used. Plant extracts, special highly active plant extract fractions and highly pure active substances isolated from plant extracts can be used. Particularly preferred are extracts, fractions and active substances from chamomile, aloe vera, commiphora species, rubia species, echinacea species, ginger, willow, willowherb, oats, black and green tea, gingko, coffee, pepper, redcurrent, tomato, vanilla, almonds, as well as pure substances such as inter alia bisabolol, apigenin-7-glucoside, boswellic acid, phytosterols, glycyrrhizinic acid, glabridin or licochalcone A.

Particularly preferred within the meaning of the invention are bisabolol, panthenol, ginger extracts and mixtures thereof. Also preferred are boswellic acid and extracts and isolated highly pure active ingredients from oats (e.g. avenanthramides) and echinacea.

Particularly preferred is α-bisabolol.

As a pseudoceramide in the processes and uses according to the invention it is preferable to use a pseudoceramide selected from the group comprising: 1,3-bis-(N-(2-hydroxyethyl)alkylamino)-2-hydroxypropanes, N-(2-hydroxyethyl)-3-oxo-2-alkyl alkylamides, N-acyl hydroxyamino acid esters and mixtures thereof. Particularly preferred is the use of a pseudoceramide selected from the group comprising: 1,3-bis-(N-(2-hydroxyethyl)palmitoylamino)-2-hydroxypropane, N-(2-hydroxyethyl)-3-oxo-2-tetradecyl octadecanamide, N-acyl hydroxyamino acid esters and mixtures thereof.

Most particularly preferred is the combination of a pseudoceramide which is an N-acyl hydroxyamino acid ester with the anti-irritant bisabolol.

If an N-acyl hydroxyamino acid ester is used as the pseudoceramide in the processes and uses according to the invention, it is preferably one having the following formula I,

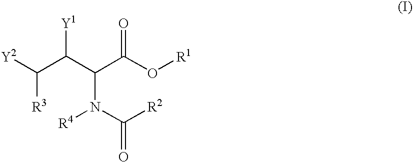

wherein
R¹ is a linear, branched or cyclic alkyl or alkenyl group having 5 to 50 carbon atoms, which is optionally substituted with one or more hydroxyl radicals,
R² is a linear or branched alkyl or alkenyl group having 1 to 49 carbon atoms, which is optionally substituted with one or more hydroxyl radicals,
Y¹ and Y² are mutually independently hydrogen or hydroxyl,
R³ and R⁴ either mutually independently stand for hydrogen or linear or branched alkyl or alkenyl groups having 1 to 10 carbon atoms or
R³ and R⁴ together represent an alkylene radical having 1 to 3 carbon atoms and together with the chain between R³ and R⁴ form a 5-, 6- or 7-membered heterocyclic ring, wherein for its part this alkylene radical is optionally substituted by 1 to 3 linear or branched alkyl or alkenyl groups or by 1 to 3 hydroxyl radicals.

Processes or uses according to the invention are preferred here wherein R¹ is a linear, branched or cyclic alkyl or alkenyl group having 5 to 24 carbon atoms, which is optionally substituted with 1 to 6 hydroxyl radicals.

R²—particularly if R¹ has the preferred meaning—is preferably a linear or branched alkyl or alkenyl group having 2 to 23 carbon atoms, which is optionally substituted with 1 to 6 hydroxyl radicals (preferably 1 to 3 hydroxyl radicals).

One of the two groups Y¹ and Y² preferably denotes a hydroxyl radical and the other a hydrogen atom.

Particularly preferred are processes or uses according to the invention wherein N-acyl hydroxyamino acid esters having formula I are used, wherein
R³ and R⁴ mutually independently stand for hydrogen or linear or branched alkyl or alkenyl groups having 1, 2, 3 or 4 carbon atoms or
R³ and R⁴ together stand for the alkylene radicals —CH₂—, —CH₂—CH₂—, —CH(OH)—, —CH(OH)—CH₂— or —CH₂—CH(OH)—.

By preference, R³ and R⁴ are hydrogen atoms and at the same time Y¹ and Y² are mutually independently hydrogen atoms or hydroxyl radicals, or

R³ and R⁴ together represent a —CH₂— or a —CH(OH)— group and together with the chain between R³ and R⁴ form a 5-membered heterocyclic ring and at the same time Y¹ and Y² are hydrogen atoms or hydroxyl radicals.

In particularly preferred cases R³ and R⁴ are hydrogen atoms and at the same time Y¹ represents a hydroxyl radical and Y² a hydrogen atom (N-acyl threonine alkyl ester) or

R³ and R⁴ together represent a —CH₂— group and together with the chain between R³ and R⁴ form a 5-membered heterocyclic ring and one of the two radicals Y¹ and Y² represents a hydroxyl radical (N-acyl hydroxyproline ester).

With regard to the N-acyl threonine alkyl esters and N-acyl hydroxyproline esters which are particularly preferably used according to the invention, R¹ preferably denotes an unbranched alkyl or alkenyl radical having 5 to 24 carbon atoms and R² preferably denotes an unbranched alkyl or alkenyl radical having 2 to 23 carbon atoms. N-Acyl threonine alkyl esters and N-acyl hydroxyproline esters having a structure of this type are amphiphilic compounds, which can be incorporated particularly well into the double membrane of the lipid barrier of the skin so that in combination with the anti-irritants for use according to the invention a particularly effective strengthening of the barrier function of undamaged skin is achieved.

The mixture for use according to the invention is produced by conventional processes known per se, such that for example one or more of the ceramides and/or pseudoceramides and anti-irritants used according to the invention are incorporated into cosmetic or dermatological formulations having a conventional composition. In addition to its skin barrier-strengthening effect, the finished mixture can also be used for example for the treatment, care or cleansing of the skin or hair or as a makeup product in decorative cosmetics.

The present invention accordingly also concerns the use (in particular) of topical cosmetic or therapeutic mixtures (formulations). Preferred mixtures preferably contain 0.01 wt. % to 30 wt. %, preferably 0.01 to 20 wt. %, but in particular 0.05 wt. % to 5 wt. %, relative to the total weight of the formulation, of the mixture components ceramide, pseudoceramide and anti-irritant for use according to the invention and can take the form of soap, synthetic detergent, liquid washing, shower and bath preparation, emulsion (as a solution, dispersion, suspension; cream, lotion or milk depending on the production process and ingredients as a W/O, O/W or multiple emulsion, PIT emulsion, emulsion foam, micro-emulsion, nano-emulsion, Pickering emulsion), as an ointment, paste, gel (including hydrogel, hydrodispersion gel, oleogel), oil, toner, balsam, serum, powder, eau de toilette, toilette, eau de cologne, perfume, wax, as a stick, roll-on, (pump) spray, aerosol (foaming, non-foaming or post-foaming), as a foot care product (including keratolytics, deodorants), as a shaving foam or aftershave (balm, lotion) as a depilatory product, hair care product such as e.g. shampoo (including 2-in-1 shampoo), conditioner, hair tonic, hair water, hair rinse, hair cream, pomade, perm and setting lotion, hair smoothing product (detangling product, relaxer), hair strengthener, styling aid (e.g. gel or wax); blonding product, hair dye (e.g. temporary hair dyes, colour rinses, semi-permanent and permanent hair dyes), as nail care products such as e.g. nail polish and nail polish remover, as deodorants and/or antiperspirants; mouthwash, makeup, makeup remover, decorative cosmetics (e.g. powder, eyeshadows, kohl pencil, lipstick).

It is also advantageous to administer the mixture for use according to the invention in encapsulated form, e.g. in gelatine, wax materials, liposomes, cellulose or cyclodextrin capsules.

Other conventional cosmetic auxiliary substances and additives can advantageously be included in the mixtures for use according to the invention in quantities of 5 to 99 wt. %, preferably 10 to 80 wt. %, relative to the total weight of the mixture. The mixtures can also have water in a quantity of up to 99.99 wt. %, preferably 5 to 80 wt. %, relative to the total weight of the formulation.

The amount of ceramides and/or pseudoceramides (one or more compounds) in the mixtures for use according to the invention is preferably 0.001 to 60 wt. %, particularly preferably 0.03 to 10 wt. %, in particular 0.05 to 5 wt. %, relative to the total weight of the mixture.

The amount of anti-irritants (one or more compounds) in the mixtures for use according to the invention is preferably 0.0001 to 20 wt. %, particularly preferably 0.001 to 10 wt. %, in particular 0.05 to 5 wt. %, relative to the total weight of the mixture.

The ratio of (a) ceramides and/or pseudoceramides to (b) anti-irritants in the mixtures for use according to the invention is conventionally (0.01-60 wt. %): (0.001-20 wt. %), preferably (0.03-20 wt. %): (0.01-10 wt. %), particularly preferably (0.05-5 wt. %): (0.05-5 wt. %).

The mixture for use according to the invention can contain cosmetic auxiliary substances and additives such as are conventionally used in cosmetic preparations, e.g. sunscreens, preservatives, bactericides, fungicides, virucides, cooling agents, insect repellents (e.g. DEET, IR 3225, Dragorepel), plant extracts, anti-inflammatory agents, substances to accelerate wound healing (e.g. chitin or chitosan and derivatives thereof, film-forming substances (e.g. polyvinyl pyrrolidones or chitosan or derivatives thereof, conventional anti-oxidants, vitamins (e.g. vitamin C and derivatives, tocopherols and derivatives, vitamin A and derivatives), 2-hydroxycarboxylic acids (e.g. citric acid, malic acid, L-, D- or di-lactic acid), skin colouring agents (e.g. walnut extracts or dihydroxyacetone), agents to promote hair growth (e.g. minoxidil, diphencyprone, hormones, finasteride, phytosterols such as e.g. β-sitosterol, biotin or extracts of Cimicifuga racemosa, Eugenia caryophyllata or Hibiscus rosasinensis, barley, hops, hydrolysates of rice or wheat), skin conditioning agents (e.g. cholesterol, ceramides, pseudoceramides), softening, moisturising or moisture-retaining substances (e.g. glycerine or urea), fats, oils, saturated fatty acids, monounsaturated or polyunsaturated fatty acids, a-hydroxy acids, polyhydroxy fatty acids or derivatives thereof (e.g. linoleic acid, α-linolenic acid, γ-linolenic acid or arachidonic acid and the natural or synthetic esters thereof, waxes or other conventional constituents of a cosmetic or dermatological formulation such as alcohols, polyols, polymers, foam stabilisers, electrolytes, organic solvents, silicone derivatives or chelating agents (e.g. ethylene diamine tetraacetic acid and derivatives), anti-dandruff agents (e.g. climbazole, ketoconazole, piroctone oleamine, zinc pyrithione), hair conditioning agents, perfumes, substances to prevent foaming, dyes, pigments having a colouring action, thickeners (advantageously silicon dioxide, aluminium silicates, such as e.g. bentonites, polysaccharides or derivatives thereof, e.g. hyaluric acid, guar gum, xanthan gum, hydroxypropyl methylcellulose or allulose derivatives, particularly advantageously polyacrylates such as e.g. carbopols or polyurethanes), surface-active substances, emulsifiers, plant parts and plant extracts (e.g. arnica, aloe, beard lichen, ivy, stinging nettle, ginseng, henna, camomile, marigold, rosemary, sage, horsetail or thyme), animal extracts such as e.g. royal jelly, propolis, proteins, protein hydrolysates, yeast extracts, hop and wheat extracts, peptides or thymus extracts.

The amounts of cosmetic or dermatological auxiliary agents and additives and perfume to be used in each case can easily be determined by the person skilled in the art by trial and error, depending on the nature of the particular product.

The mixture for use according to the invention advantageously contains at least one UVA filter and/or at least one UVB filter and/or at least one inorganic pigment. The mixtures can be in various forms, such as are conventionally used for example for sunscreen preparations to protect the skin and hair against ultraviolet radiation. Thus for example they can form a solution, a water-in-oil (W/O) or oil-in-water (O/W) emulsion, or a multiple emulsion, of the water-in-oil-in-water (W/O/W) type for example, a gel, a hydrodispersion, a solid stick or an aerosol. The total amount of filter substances here is 0.01 wt. % to 40 wt. %, preferably 0.1% to 10 wt. %, in particular 1.0 to 5.0 wt. %, relative to the total weight of the mixture, to provide cosmetic mixtures (preparations).

Advantageous UV filters are, for example:
p-aminobenzoic acid
p-aminobenzoic acid ethyl ester (25 mol) ethoxylated
p-dimethylaminobenzoic acid-2-ethylhexyl ester
p-aminobenzoic acid ethyl ester (2 mol) N-propoxylated
p-aminobenzoic acid glycerol ester
salicylic acid homomethyl ester (homosalates) (Neo Heliopan®HMS)
salicylic acid-2-ethylhexyl ester (Neo Heliopan®OS)
triethanolamine salicylate
4-isopropyl benzyl salicylate
anthranilic acid menthyl ester (Neo Heliopan®MA)
diisopropyl cinnamic acid ethyl ester
p-methoxycinnamic acid-2-ethylhexyl ester (Neo Heliopan®AV)
diisopropyl cinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-ethylhexyl-2-cyano-3,3-diphenyl acrylate (Neo Heliopan®303)
ethyl-2-cyano-3,3′-diphenyl acrylate
2-phenylbenzimidazole sulfonic acid and salts (Neo Heliopan®Hydro)
3-(4′-trimethylammonium)benzylidene bornan-2-one methyl sulfate
terephthalylidene dibornane sulfonic acid and salts (Mexoryl®SX)
4-t-butyl-4′-methoxydibenzoyl methane (avobenzone)/(Neo Heliopan®357)
β-imidazole-4(5)-acrylic acid (urocanic acid)
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methyl benzophenone
3-(4′-sulfo)benzylidene bornan-2-one and salts
3-(4′-methyl benzylidene)-d,l-camphor (Neo Heliopan®MBC)
3-benzylidene-d,l-camphor
4-isopropyl dibenzoyl methane
2,4,6-trianilino-(p-carbo-2′-ethylhexyl-1′-oxy)-1,3,5-triazine
phenylene bis-benzimidazyl tetrasulfonic acid disodium salt (Neo Heliopan®AP)
2,2′-(1,4-phenylene)-bis-(1H-benzimidazole-4,6-disulfonic acid), monosodium salt
N-[(2 and 4)-[2-(oxoborn-3-ylidene)methyl]benzyl]acrylamide polymer
phenol, -(2H-benzotriazol-2-yl)-4-methyl-6-(2-methyl-3(1,3,3,3-tetramethyl-1-(trimethylsilyl)oxy)disiloxyanyl)propyl), (Mexoryl®XL)
4,4′-[(6-[4-(1,1-dimethyl)aminocarbonyl)phenylamino]-1,3,5-triazine-2,4-diyl)diimino]-bis-(benzoic acid-2-ethylhexyl ester) (Uvasorb®HEB)
2,2′-methylene bis-(6-(2H-benzotriazol-2-yl)-4-1,1,3,3-tetramethylbutyl)phenol), (Tinosorb®M)
2,4-bis-[4-(2-ethylhexyloxy)-2-hydroxyphenyl]-1,3,5-triazine
benzylidene malonate polysiloxane (Parsol®SLX)
glyceryl ethylhexanoate dimethoxycinnamate
disodium-2,2′-dihydroxy-4,4′-dimethoxy-5,5′-disulfobenzophenone
dipropylene glycol salicylate
sodium hydroxymethoxybenzophenone sulfonate
4,4′,4-(1,3,5-triazine-2,4,6-triyltriimino)-tris-benzoic acid tris(2-ethylhexyl ester) (Uvinul®T150)
2,4-bis-[{(4-(2-ethyl hexyloxy)-2-hydroxy}phenyl]-6-(4-methoxyphenyl)-1,3,5-triazine (Tinosorb®S)
2,4-bis-[{(4-(3-sulfonato)-2-hydroxypropyloxy)-2-hydroxy}phenyl]-6-(4-methoxyphenyl)-1,3,5-triazine sodium salt
2,4-bis-[{(3-(2-propyloxy)-2-hydroxypropyloxy)-2-hydroxy}phenyl]-6-(4-methoxyphenyl)-1,3,5-triazine
2,4-bis-[{4-(2-ethylhexyloxy)-2-hydroxy}phenyl]-6-[4-(2-methoxyethyl carbonyl)phenylamino]-1,3,5-triazine
2,4-bis-[{4-(3-(2-propyloxy)-2-hydroxypropyloxy)-2-hydroxy}phenyl]-6-[4-(2-ethylcarboxyl)phenylamino]-1,3,5-triazine
2,4-bis-[{4-(2-ethylhexyloxy)-2-hydroxy}phenyl]-6-(1-methylpyrrol-2-yl)-1,3,5-triazine
2,4-bis-[{-4-tris-(trimethylsiloxysilylpropyloxy)-2-hydroxy}phenyl]-6-(4-methoxyphenyl)-1,3,5-triazine
2,4-bis-[{4-(2″-methylpropenyloxy)-2-hydroxy}phenyl]-6-(4-methoxyphenyl)-1,3,5-triazine
2,4-bis-[{4-(1′,1′,1′,3′5′,5′,5′-heptamethylsiloxy-2″-methylpropyloxy)-2-hydroxy}phenyl]-6-(4-methoxyphenyl)-1,3,5-triazine
2-(4-diethylamino-2-hydroxybenzoyl)benzoic acid hexyl ester (Uvinul® A Plus)
indanylidene compounds in accordance with DE 100 55 940 (=WO 02/38537)

UV absorbers which are particularly suitable for combining are
p-aminobenzoic acid
3-(4′-trimethylammonium) benzylidene bornan-2-one methyl sulfate
salicylic acid homomethyl ester (Neo Heliopan®HMS)
2-hydroxy-4-methoxybenzophenone (Neo Heliopan®BB)
2-phenylbenzimidazole sulfonic acid (Neo Heliopan®Hydro)
terephthalylidene dibornane sulfonic acid and salts (Mexoryl®SX)
4-tert-butyl-4′-methoxydibenzoyl methane (Neo Heliopan®357)
3-(4′-sulfo)benzylidene bornan-2-one and salts
2-ethylhexyl-2-cyano-3,3-diphenyl acrylate (Neo Heliopan®303)
N-[(2 and 4)-[2-(oxoborn-3-ylidene)methyl]benzyl]acrylamide polymer
p-methoxycinnamic acid-2-ethylhexyl ester (Neo Heliopan®AV)
p-aminobenzoic acid ethyl ester (25 mol) ethoxylated
p-methoxycinnamic acid isoamyl ester (Neo Heliopan®E1000)
2,4,6-trianilino-(p-carbo-2′-ethylhexyl-1′-oxy)-1,3,5-triazine (Uvinul®T150)
phenol, 2-(2H-benzotriazol-2-yl)-4-methyl-6-(2-methyl-3(1,3,3,3-tetramethyl-1-(trimethylsilyl)oxy)disiloxyanyl)propyl), (Mexoryl®XL)
4,4′-[(6-[4-(1,1-dimethyl)aminocarbonyl)phenylamino]-1,3,5-triazine-2,4-diyl)diimino]-bis-(benzoic acid-2-ethylhexyl ester) (Uvasorb HEB)
3-(4′-methyl benzylidene)-d,l-camphor (Neo Heliopan®MBC)
3-benzylidene camphor
salicylic acid-2-ethylhexyl ester (Neo Heliopan®OS)
4-dimethylaminobenzoic acid-2-ethylhexyl ester (Padimate O)
hydroxy-4-methoxybenzophenone-5-sulfonic acid and Na salt
2,2′-methylene bis-(6-(2H-benzotriazol-2-yl)-4-1,1,3,3-tetramethylbutyl)phenol) (Tinosorb®M)
phenylene bis-benzimidazyl tetrasulfonic acid disodium salt (Neo Heliopan®AP)
2,4-bis-[{(4-(2-ethyl hexyloxy)-2-hydroxy}phenyl]-6-(4-methoxyphenyl)-1,3,5-triazine, (Tinosorb®S)
benzylidene malonate polysiloxane (Parsol®SLX)
menthyl anthranilate (Neo Heliopan®MA)
2-(4-diethylamino-2-hydroxybenzoyl)benzoic acid hexyl ester (Uvinul® A Plus)
indanylidene compounds in accordance with DE 100 55 940 (=WO 02/38537)

Advantageous inorganic light protection pigments are finely dispersed metal oxides and metal salts, for example titanium dioxides, zinc oxide (ZnO), iron oxides (e.g. Fe₂O₃), aluminium oxide (Al₂O₃); cerium oxides (e.g. Ce₂O₃), manganese oxides (e.g. MnO), zirconium oxide (ZrO 2), silicon oxide (SiO₂), mixed oxides of the corresponding metals and mixtures of such oxides, barium sulfate and zinc stearate. Pigments based on TiO₂ or zinc oxide are particularly preferred. In preferred embodiments the particles have an average diameter of less than 100 nm, preferably between 5 and 50 nm and particularly preferably between 15 and 30 nm. They can display a spherical form, but such particles having an ellipsoid form or other form deviating from the spherical shape can also be used. The pigments can also be surface treated, i.e. hydrophilised or hydrophobed. Typical examples are coated titanium dioxides, such as e.g. titanium dioxide T 805 (Degussa) or Eusolex® T2000 (Merck) or coated zinc oxide, such as e.g. zinc oxide NDM. Suitable hydrophobic coating agents are above all silicones and especially trialkoxyoctyl silanes or simethicones. So-called micro-pigments or nano-pigments are preferably used in sunscreens. Zinc micro- or nano-pigments are preferably used.

The total amount of inorganic pigments, particularly hydrophobic inorganic micro-pigments, in the finished cosmetic or dermatological formulations is advantageously in the range from 0.1 to 30 wt. %, preferably 0.1 to 10.0, in particular 0.5 to 6.0 wt. %, relative to the total weight of the formulations.

The mixtures for use according to the invention can also contain antioxidants, wherein all antioxidants that are suitable for or commonly used for cosmetic and/or dermatological applications can be used. The antioxidants are advantageously selected from the group consisting of amino acids (e.g. glycine, histidine, tyrosine, tryptophane) and derivatives thereof, imidazoles (e.g. urocanic acid) and derivatives thereof, peptides such as D, L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propyl thiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and the salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximine, homocysteine sulfoximine, buthionine sulfone, penta-, hexa-, heptathionine sulfoximine) in very small compatible doses, also (metal) chelators, e.g. α-hydroxy fatty acids, palmitic acid, phytic acid, lactoferrin, α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate, ascorbyl glycosides such as e.g. 6-O-acyl-2-O-α-D-glucopyranosyl-L-ascorbic acid, 6-O-acyl-2-O-β-D-glucopyranosyl-L-ascorbic acid, 2-O-α-D-glucopyranosyl-L-ascorbic acid or 2-O-β-D-glucopyranosyl-L-ascorbic acid), tocopherols and derivatives thereof (e.g. vitamin E acetate), vitamin A and derivatives thereof (vitamin A palmitate) as well as coniferyl benzoate of benzoic resin, rutic acid and derivatives thereof, a-glucosyl rutin, quercetin and derivatives thereof, rosemarinic acid, carnosol, carnosolic acid, resveratrol, caffeic acid and derivatives thereof, sinapic acid and derivatives thereof, ferulic acid and derivatives thereof, furfurylidene glucitol, curcuminoids, butyl hydroxytoluene, butyl hydroxyanisole, nordihydroguaiacic resin acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄) selenium and derivatives thereof (e.g. selenium methionine), stilbenes and derivatives thereof (e.g. stilbene oxide, trans-stilbene oxide) along with derivatives (salts, esters, ethers, sugars, nucleotides, nucleosides, peptides and lipids) of these cited active ingredients or extracts or fractions of plants having an antioxidant effect, such as e.g. green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort.

The amount of antioxidants (one or more compounds) in the mixtures for use according to the invention is preferably 0.01 to 20 wt. %, particularly preferably 0.05 to 10 wt. %, in particular 0.2 to 5 wt. %, relative to the total weight of the preparation.

If vitamin E and/or derivatives thereof are used as the antioxidant(s), it is advantageous to choose their concentrations from the range from 0.001 to 10 wt. %, relative to the total weight of the formulation.

If vitamin A or vitamin A derivatives or carotenes or derivatives thereof are used as the antioxidant(s), it is advantageous to choose their concentrations from the range from 0.001 to 10 wt. %, relative to the total weight of the formulation.

The (cosmetic) mixtures for use according to the invention can also contain active ingredients and combinations of active ingredients to combat skin ageing and wrinkles. All active ingredients that are suitable for or commonly used for cosmetic and/or dermatological applications to combat skin ageing and wrinkles can be used here according to the invention. Advantageous active ingredients in this respect to combat skin ageing and wrinkles are soya protein or protein hydrolysates, soya isoflavones, hydrolysed rice protein, hydrolysed hazelnut protein, oligopeptides from hydrolysed Hibiscus esculentus extract, wheat protein, β-glucanes e.g. from oats and derivatives thereof, glycoproteins, ursolic acid and salts thereof, betulin, betulinic acid and salts thereof, retinol, retinol palmitate, propyl gallate, precocene, 6-hydroxy-7-methoxy-2,2-dimethyl-1(2H)-benzopyran, 3,4-dihydro-6-hydroxy-7-methoxy-2,2-dimethyl-1(2H)-benzopyran, creatine or other synthetic or natural active ingredients to combat skin ageing and wrinkles, wherein the latter can also be used in the form of an extract from plants, such as e.g. green tea, Rubus fruticosus, Sanguisorba officinalis, Centella asiatica, Ribes nigrum, Passiflora incarnate, Phyllanthus emblica, okra, algae, evening primrose, rosemary, sage, echinacea, birch, apple or soya.

Particularly preferred for use as additional active ingredients to combat skin ageing is β-glucane, wherein 1,3-1,4-coupled β-glucane from oats, Rubus fruticosus extract or wheat protein is especially preferred.

Mixtures for use according to the invention in the form of a cosmetic preparation can advantageously also contain moisture regulators. The following substances, for example, can be used as moisture regulators (moisturisers): sodium lactate, urea, urea derivatives, alcohols, glycerol, diols such as propylene glycol, 1,2-pentanediol, 1,2-hexanediol and 1,2-octanediol, collagen, elastin or hyaluric acid, diacyl adipates, petroleum jelly, urocanic acid, lecithin, panthenol, phytanetriol, lycopene, (pseudo)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides such as e.g. glucose, galactose, fructose, mannose, fruit sugars and lactose, poly sugars such as β-glucanes, in particular 1,3-1,4-β-glucane from oats, alpha-hydroxy fatty acids, triterpene acids such as betulinic acid or ursolic acid, algal extracts.

A mixture for use according to the invention can also be used together with osmolytes. Examples of osmolytes which can be cited are: substances from the group of sugar alcohols (myo-inositol, mannitol, sorbitol), quaternary amines such as taurine, choline, betaine, betaine glycine, ectoine, diglycerol phosphate, phosphorylcholine, glycerophosphorylcholines, amino acids such as glutamine, glycine, alanine, glutamate, aspartate or proline, phosphatidylcholine, phosphatidylinositol, inorganic phosphates, and polymers of the cited compounds such as proteins, peptides, polyamino acids and polyols. All osmolytes also have a skin-moistening action.

The mixtures for use according to the invention can preferably also contain active ingredients which stimulate skin and hair tinting or tanning by chemical or natural means. A more rapid action based on synergistic effects is achieved in this way. Particularly preferred here are substrates or substrate analogues of tyrosinase such as L-tyrosine, L-DOPA or L-dihydroxyphenylalanine, stimulators of tyrosinase activity or expression such as theophylline, caffeine, proopiomelanocortin peptides such as ACTH, alpha-MSH, peptide analogues thereof and other substances which bind to the melanocortin receptor, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes into keratinocytes such as serine proteases or agonists of the PAR-2 receptor, extracts of plants and plant parts of the chrysanthemum species, sanguisorba species, walnut extracts, urucum extracts, rhubarb extracts, erythrulose and dihydroxyacetone.

The mixture for use according to the invention can in many cases advantageously be used in combination with skin lightening active ingredients. All skin-lightening active ingredients that are suitable for or commonly used for cosmetic and/or dermatological applications can be used here according to the invention. Advantageous skin-lightening active ingredients in this respect are kojic acid (5-hydroxy-2-hydroxymethyl-4-pyranone), kojic acid derivatives e.g. kojic acid dipalmitate, arbutin, ascorbic acid, ascorbic acid derivatives, hydroquinone, hydroquinone derivatives, resorcinol, sulfur-containing molecules such as e.g. glutathione or cysteine, alpha-hydroxy acids (e.g. citric acid, lactic acid, malic acid) and derivatives thereof, N-acetyl tyrosine and derivatives, undecenoyl phenylalanine, gluconic acid, 4-alkyl resorcinols, chromone derivatives such as aloesin, flavonoids, thymol derivatives, 1-aminoethyl phosphinic acid, thio urea derivatives, ellagic acid, nicotinamide, zinc salts such as e.g. zinc chloride or gluconate, thujaplicin and derivatives, triterpenes such as maslinic acid, sterols such as ergosterol, benzofuranones such as senkyunolide, vinyl and ethyl guiacol, inhibitors of nitrogen oxide synthesis, such as e.g. L-nitroarginine and derivatives thereof, 2,7-dinitroindazole or thiocitrulline, metal chelators (e.g. α-hydroxy fatty acids, palmitic acid, phytic acid, lactoferrin, humic acid, bile acid, bile extracts, bilirubin, biliverdin, EDTA, EGTA and derivatives thereof, retinoids, soya milk, serine protease inhibitors or lipoic acid or other synthetic or natural active ingredients for skin and hair lightening, wherein the latter can also be used in the form of an extract from plants, such as e.g. bearberry extract, rice extract, liquorice root extract or constituents concentrated therefrom, such as glabridin or licochalcone A, artocarpus extract, extract from rumex and ramulus species, extracts from pine species (pinus) and extracts from vitis species or stilbene derivatives concentrated therefrom, extract of saxifrage, mulberry, scutelleria or/and grapes.

Mixtures for use according to the invention in the form of cosmetic preparations can also contain anionic, cationic, non-ionic and/or amphoteric surfactants, especially if crystalline or microcrystalline solids, for example inorganic micropigments, are to be incorporated into the mixtures.

Anionic surfactants generally display carboxylate, sulfate or sulfonate groups as functional groups. In aqueous solution they form negatively charged organic ions in the acid or neutral environment. Cationic surfactants are almost exclusively characterised by the presence of a quaternary ammonium group. In aqueous solution they form positively charged organic ions in the acid or neutral environment. Amphoteric surfactants contain both anionic and cationic groups and therefore behave in aqueous solution in the same way as anionic or cationic surfactants, depending on the pH. They have a positive charge in a strongly acid environment and a negative charge in an alkaline environment. In the neutral pH range, by contrast, they are zwitterionic. Polyether chains are typical of non-ionic surfactants. Non-ionic surfactants do not form ions in the aqueous medium.

### A. Anionic Surfactants

Anionic surfactants which can advantageously be used are acyl amino acids (and salts thereof), such as
acyl glutamates, for example sodium acyl glutamate, di-TEA-palmitoyl aspartate and sodium caprylic/capric glutamate,
acyl peptides, for example palmitoyl-hydrolysed milk protein, sodium cocoyl-hydrolysed soya protein and sodium/potassium cocoyl-hydrolysed collagen,
sarcosinates, for example myristoyl sarcosin, TEA-lauroyl sarcosinate, sodium lauroyl sarcosinate and sodium cocoyl sarcosinate,
taurates, for example sodium lauroyl taurate and sodium methyl cocoyl taurate,
acyl lactylates, lauroyl lactylate, caproyl lactylate
alaninates
   carboxylic acid and derivatives, such as
   for example lauric acid, aluminium stearate, magnesium alkanolate and zinc undecylenate,
ester carboxylic acids, for example calcium stearoyl lactylate, laureth-6 citrate and sodium PEG-4 lauramide carboxylate,
ether carboxylic acids, for example sodium laureth-13 carboxylate and sodium PEG-6 cocamide carboxylate,
   phosphoric acid esters and salts, such as e.g. DEA-oleth-10-phosphate and dilaureth-4 phosphate,
   sulfonic acids and salts, such as
acyl isothionates, e.g. sodium/ammonium cocoyl isethionate,
alkyl aryl sulfonates,
alkyl sulfonates, for example sodium cocomonoglyceride sulfate, sodium C₁₂₋₁₄ olefin sulfonate, sodium lauryl sulfoacetate and magnesium PEG-3 cocamide sulfate,
sulfosuccinates, for example dioctyl sodium sulfosuccinate, disodium laureth sulfosuccinate, disodium lauryl sulfosuccinate and disodium undecylenamido MEA sulfosuccinate
   and
   sulfuric acid esters, such as
alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

### B. Cationic Surfactants

Cationic surfactants which can advantageously be used are
alkyl amines,
alkyl imidazoles,
ethoxylated amines and
quaternary surfactants.
   RNH₂CH₂CH₂COO⁻ (where pH=7)
   RNHCH₂CH₂COO—B⁺ (where pH=12) B⁺=any cation, e.g. Na⁺
esterquats

Quaternary surfactants contain at least one N atom, which is covalently bonded to 4 alkyl or aryl groups. This leads to a positive charge, regardless of the pH. Alkyl betaine, alkyl amidopropyl betaine and alkyl amidopropyl hydroxysulfaine are advantageous. The cationic surfactants used can also preferably be chosen from the group of quaternary ammonium compounds, in particular benzyl trialkyl ammonium chlorides or bromides, such as benzyl dimethylstearyl ammonium chloride for example, also alkyl trialkyl ammonium salts, for example cetyl trimethyl ammonium chloride or bromide, alkyl dimethyl hydroxyethyl ammonium chlorides or bromides, dialkyl dimethyl ammonium chlorides or bromides, alkyl amide ethyl trimethyl ammonium ether sulfates, alkyl pyridinium salts, for example lauryl or cetyl pyrimidinium chloride, imidazoline derivatives and compounds having a cationic character such as amine oxides, for example alkyl dimethyl amine oxides or alkyl aminoethyl dimethyl amine oxides. Cetyl trimethyl ammonium salts are particularly advantageously used.

### C. Amphoteric Surfactants

Amphoteric surfactants which can advantageously be used are
acyl/dialkyl ethylene diamine, for example sodium acyl amphoacetate, disodium acyl amphodipropionate, disodium alkyl amphodiacetate, sodium acyl amphohydroxypropyl sulfonate, disodium acyl amphodiacetate and sodium acyl amphopropionate,
N-alkyl amino acids, for example aminopropyl alkyl glutamide, alkyl aminopropionic acid, sodium alkyl imidodipropionate and lauroamphocarboxyglycinate.

### D. Non-Ionic Surfactants

Non-ionic surfactants which can advantageously be used are
alcohols,
alkanolamides, such as cocamides MEA/DEA/MIPA,
amine oxides, such as cocamidopropylamine oxide,
esters produced by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides such as lauryl glucoside, decyl glycoside and cocoglycoside.
sucrose esters, ethers
polyglycerol esters, diglycerol esters, monoglycerol esters
methyl glucose esters, esters of hydroxy acids

The use of a combination of anionic and/or amphoteric surfactants with one or more non-ionic surfactants is also advantageous.

The surface-active substance can be present in the mixtures for use according to the invention in a concentration of between 1 and 98 wt. %, relative to the total weight of the mixture.

A lipid phase in mixtures for use according to the invention can advantageously be chosen from the following groups of substances:
mineral oils (advantageously paraffin oil), mineral waxes
fatty oils, fats, waxes and other natural and synthetic fat bodies, preferably esters of fatty acids with low C-number alcohols, for example with isopropanol, propylene glycol or glycerol, or esters of fatty alcohols with low C-number alkanoic acids or with fatty acids;
alkyl benzoates;
silicone oils such as dimethyl polysiloxanes, diethyl polysiloxanes, diphenyl polysiloxanes and mixed forms thereof
hydrocarbons (advantageously squalane or squalene)
synthetic or semisynthetic triglyceride oils (e.g. triglycerides of capric or caprylic acid)
natural oils (one or more conditioning animal and/or vegetable fats and oils such as olive oil, sunflower oil, refined soya oil, palm oil, sesame oil, rapeseed oil, almond oil, borage oil, evening primrose oil, coconut butter, shea butter, jojoba oil, oat oil, sperm oil, beef fat, neatsfoot oil and pig fat)
   and optionally other conditioning constituents such as e.g. fatty alcohols having 8-30 C atoms. The fatty alcohols here can be saturated or unsaturated and linear or branched. Examples that can be used include decanol, decenol, octanol, octenol, dodecanol, octadienol, decadienol, dodecadienol, oleyl alcohol, ricinol alcohol, erucic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as Guerbet alcohols thereof, wherein the list could be extended almost at will with other alcohols having a related chemical structure. The fatty alcohols preferably come from natural fatty acids, being conventionally produced from the corresponding esters of the fatty acids by reduction. Also usable are fatty alcohol fractions produced by reduction from naturally occurring fats and fatty oils, such as e.g. beef fat, groundnut oil, colza oil, cottonseed oil, soya bean oil, sunflower oil, palm kernel oil, linseed oil, maize oil, castor oil, rapeseed oil, sesame oil, cocoa butter and coconut butter. Synthetic ester oils can also be included. Preferred examples are esters of saturated and/or unsaturated, linear and/or branched alkane carboxylic acids having 3 to 30 C atoms with saturated and/or unsaturated, linear and/or branched alcohols having 3 to 30 C atoms and esters of aromatic carboxylic acids with saturated and/or unsaturated, linear and/or branched alcohols having 3 to 30 atoms, selected in particular from the group comprising isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laurate, 2-ethylhexyl ethylhexanoate, cetearyl-2-ethylhexanoate, 3,5,5-trimethylhexyl-3,5,5-trimethyl hexanoate, 2-ethylhexyl isononanoate, 2-ethylhexyl-3,5,5-trimethyl hexanoate, 2-ethylhexyl-2-ethylhexanoate, 2-hexyl decyl stearate, 2-octyl decyl palmitate, oleyl oleate, oleyl erucate, erucyl oleate, erucyl erucate and synthetic or natural mixtures of such esters), fats, waxes and other natural and synthetic fat bodies, preferably esters of fatty alcohols with low C-number alcohols (e.g. with isopropanol, propylene glycol or glycerol) or esters of fatty alcohols with low C-number alkanoic acids or with fatty acids, alkyl benzoates (e.g. mixtures of n-dodecyl, n-tridecyl, n-tetradecyl and n-pentadecyl benzoate) and cyclic or linear silicone oils (such as e.g. dimethyl polysiloxanes, diethyl polysiloxanes, diphenyl polysiloxanes and mixed forms thereof).

Other conditioning substances which combine well with the mixture for use according to the invention include
waxes such as e.g. candelilla wax or carnauba wax
ceramides, wherein ceramides are understood to be N-acyl sphingosines (fatty acid amides of sphingosine) or synthetic analogues of such lipids (so-called pseudoceramides), which markedly improve the water-retaining capacity of the stratum corneum.
phospholipids, for example soya lecithin, egg lecithin and kephalins
vaseline, paraffin and silicone oils; the latter include inter alia dialkyl and alkylaryl siloxanes such as dimethyl polysiloxane and methylphenyl polysiloxane, as well as alkoxylated and quaternised derivatives thereof.

An aqueous phase of a mixture for use according to the invention can advantageously include: alcohols, diols or polyols having a low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols having a low C number, e.g. ethanol, isopropanol, 1,2-propanediol, glycerol and in particular one or more thickeners, which can advantageously be chosen from the group comprising silicon dioxide, aluminium silicates, polysaccharides or derivatives thereof, e.g. hyaluronic acid, xanthan gum, hydroxypropyl methyl cellulose, particularly advantageously from the group of polyacrylates, preferably a polyacrylate from the group of so-called carbopols, for example type 980, 981, 1382, 2984, 5984 carbopols, either individually or in combination.

Mixtures for use according to the invention in the form of an emulsion advantageously include one or more emulsifiers. O/W emulsifiers, for example, can advantageously be chosen from the group of polyethoxylated or polypropoxylated or polyethoxylated and polypropoxylated products, e.g.:
fatty alcohol ethoxylates
ethoxylated wool wax alcohols,
polyethylene glycol ethers having the general formula R—O—(—CH₂—CH₂—O—)ₙ—R′,
fatty acid ethoxylates having the general formula R—COO—(—CH₂—CH₂—O—)ₙ—H,
etherified fatty acid ethoxylates having the general formula

R—COO—(—CH₂—CH₂—O—)ₙ—R′,
esterified fatty acid ethoxylates having the general formula

R—COO—(—CH₂—CH₂—O—)ₙ—C(O)—R′,
polyethylene glycol glycerol fatty acid esters
ethoxylated sorbitan esters,
cholesterol ethoxylates
ethoxylated triglycerides
alkyl ether carboxylic acids having the general formula
   R—COO—(—CH₂—CH₂—O—)ₙ—OOH, where n represents a number from 5 to 30,
polyoxyethylene sorbitol fatty acid esters,
alkyl ether sulfates having the general formula R—O—(—CH₂—CH₂—O—)ₙ—SO₃—H
fatty alcohol propoxylates having the general formula R—O—(—CH₂—CH(CH₃)—O—)ₙ—H
polypropylene glycol ethers having the general formula

R—O—(—CH₂—CH(CH₃)—O—)ₙ—R′
propoxylated wool wax alcohols,
etherified fatty acid propoxylates R—COO—(—CH₂—CH(CH₃)—O—)ₙ—R′
esterified fatty acid propoxylates having the general formula

R—COO—(—CH₂—CH(CH₃)—O—)ₙ—C(O)—R′
fatty acid propoxylates having the general formula

R—COO—(—CH₂—CH(CH₃)—O—)ₙ—H,
polypropylene glycol glycerol fatty acid esters
propoxylated sorbitan esters,
cholesterol propoxylates
propoxylated triglycerides,
alkyl ether carboxylic acids having the general formula

R—O—(—CH₂—CH(CH₃)—O—)ₙ—CH₂—COOH,
alkyl ether sulfates or the acids underlying these sulfates
   having the general formula R—O—(—CH₂—CH(CH₃)—O—)ₙ—SO₃—H,
fatty alcohol ethoxylates/propoxylates having the general formula R—O—Xₙ—Yₘ—H
polypropylene glycol ethers having the general formula R—O—Xₙ—Yₘ—R′
etherified fatty acid propoxylates having the general formula R—COO—Xₙ—Yₘ—R′
fatty acid ethoxylates/propoxylates having the general formula R—COO—Xₙ—Yₘ—H.

Particularly advantageously according to the invention the polyethoxylated or polypropoxylated or polyethoxylated and polypropoxylated O/W emulsifiers used are chosen from the group of substances having HLB values of 11 to 18, most particularly advantageously having HLB values of 14.5 to 15.5, if the O/W emulsifiers display saturated R and R′ radicals. If the O/W emulsifiers display unsaturated R and/or R′ radicals, or if isoalkyl derivatives are present, the preferred HLB value of such emulsifiers can also be lower or higher.

It is advantageous to choose the fatty alcohol ethoxylates from the group of ethoxylated stearyl alcohols, cetyl alcohols, cetyl stearyl alcohols (cetearyl alcohols). Particularly preferred are:

Polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is also advantageous to choose the fatty acid ethoxylates from the following group:

Polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

Sodium laureth-11 carboxylate can advantageously be used as the ethoxylated alkyl ether carboxylic acid or its salt. Sodium laureth 1-4 sulfate can advantageously be used as the alkyl ether sulfate. Polyethylene glycol (30) cholesteryl ether can advantageously be used as the ethoxylated cholesterol derivative. Polyethylene glycol (25) soya sterol has also proved itself.

Polyethylene glycol (60) evening primrose glycerides can advantageously be used as ethoxylated triglycerides.

It is also advantageous to choose the polyethylene glycol glycerol fatty acid esters from the group comprising polyethylene glycol (20) glyceryl laurate, polyethylene glycol (21) glyceryl lau rate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise advantageous to choose the sorbitan esters from the group comprising polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be used as advantageous W/O emulsifiers: fatty alcohols having 8 to 30 carbon atoms, monoglycerol esters of saturated and/or unsaturated, branched and/or unbranched alkane carboxylic acids having a chain length of 8 to 24, in particular 12 to 18 C atoms, diglycerol esters of saturated and/or unsaturated, branched and/or unbranched alkane carboxylic acids having a chain length of 8 to 24, in particular 12 to 18 C atoms, monoglycerol ethers of saturated and/or unsaturated, branched and/or unbranched alcohols having a chain length of 8 to 24, in particular 12 to 18 C atoms, diglycerol ethers of saturated and/or unsaturated, branched and/or unbranched alcohols having a chain length of 8 to 24, in particular 12 to 18 C atoms, propylene glycol esters of saturated and/or unsaturated, branched and/or unbranched alkane carboxylic acids having a chain length of 8 to 24, in particular 12 to 18 C atoms and sorbitan esters of saturated and/or unsaturated, branched and/or unbranched alkane carboxylic acids having a chain length of 8 to 24, in particular 12 to 18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Mixtures for use according to the invention (e.g. a topical cosmetic formulation) advantageously contain cooling agents. Examples of cooling agents which can be cited are: l-menthol, d-menthol, racemic menthol, menthone glycerine acetal, menthyl lactate, substituted menthyl-3-carboxylic acid amides (e.g. menthyl-3-carboxylic acid-N-ethylamide), 2-isopropyl-N-2,3-trimethyl butanamide, substituted cyclohexane carboxylic acid amides, 3-menthoxypropane-1,2-diol, 2-hydroxyethyl menthyl carbonate, 2-hydroxypropyl menthyl carbonate, N-acetyl glycine menthyl ester, isopulegol, menthyl hydroxycarboxylic acid esters (e.g. menthyl-3-hydroxybutyrate), monomenthyl succinate, 2-mercaptocyclodecanone, menthyl-2-pyrrolidin-5-one carboxylate, 2,3-dihydroxy-p-menthane, 3,3,5-trimethyl cyclohexanone glycerine ketal, 3-menthyl-3,6-di- and trioxaalkanoates, 3-menthyl methoxyacetate, icilin.

The mixtures for use according to the invention (e.g. topical cosmetic formulations) also advantageously contain antimicrobial active ingredients. Worth mentioning in addition to standard preservatives as further active ingredients are in particular, in addition to the large group of standard antibiotics, the products relevant for cosmetics, such as triclosan, climbazole, zinc pyrithione, ichthyol, octopirox (1-hydroxy-4-methyl-6-(2,4,4-trimethylpentyl)-2(1H)-pyridones, 2-aminoethanol), chitosan, farnesol, octoxyglycerine, glycerol monolaurate, aryl alkyl alcohols such as e.g. phenylethyl alcohol, 3-phenyl-1-propanol, veticol or muguet alcohol and aliphatic diols such as e.g. 1,2-decanediol or combinations of the cited substances, which are used inter alia against underarm odour, foot odour or dandruff formation.

Aryl- or aryloxy-substituted, unbranched or monoalkyl- and polyalkyl-branched saturated or unsaturated
fatty alcohols, aldehydes and acids, acid esters
alkane diols, dialdehydes and dicarboxylic acids and esters
   having chain lengths of C₂ to C₄₀, from synthetic or natural sources (e.g. from coconut butter, palm kernel oil, wool wax, lanolin).

Monohydroxy and oligohydroxy fatty acids having chain lengths of C₂ to C₂₄ (e.g. lactic acid, 2-hydroxypalmitic acid), oligomers and/or polymers thereof and plant and animal raw materials containing these.

Ethoxylate, propoxylated or mixed ethoxylated/propoxylated cosmetic fatty alcohols, fatty acids and fatty acid esters having chain lengths of C₂ to C₄₀ having 1 to 150 E/O and/or P/O units.

So-called “natural” antibacterial active ingredients can also be used, most of which are essential oils. Typical oils having an antibacterial action are, for example, oils of aniseed, lemon, orange, rosemary, wintergreen, clove, thyme, lavender, hops, citronella, wheat, lemongrass, cedarwood, cinnamon, geranium, sandalwood, violet, eucalyptus, peppermint, gum benzoin, basil, fennel and Ocmea origanum, Hydastis carradensis, Berberidaceae daceae, Ratanhiae or Curcuma longa.

Important substances having an antimicrobial action which can be found in essential oils are for example anethol, catechol, camphene, carvacrol, eugenol, eucalyptol, ferulic acid, farnesol, hinokitiol, tropolone, limonene, menthol, methyl salicylate, thymol, terpineol, verbenone, berberine, curcumin, caryophyllene oxide, nerolodol, geraniol.

Mixtures of the cited active systems or active ingredients and active ingredient combinations containing these active ingredients can also be used.

The amount of active ingredients in the preparations is preferably 0.01 to 20 wt. %, relative to the total weight of the preparations, particularly preferably 0.05 to 10 wt. %.

A mixture for use according to the invention can moreover also be used in combination with sweat-inhibiting active ingredients (antiperspirants) and odour absorbers. Aluminium salts above all such as aluminium chloride, aluminium chlorohydrate, nitrate, sulfate, acetate, etc., but also aluminium hydroxychlorides, can be used as sweat-inhibiting active ingredients. The use of zinc, magnesium and zirconium compounds can also be advantageous, however. The following can also be used: a) protein-precipitating substances such as inter alia formaldehyde, glutaraldehyde, natural and synthetic tannins and trichloroacetic acid, which bring about a surface closure of the sweat glands, b) local anesthetics (including dilute solutions of e.g. lidocaine, prilocaine or mixtures of such substances), which switch off the sympathic supply to the sweat glands by blocking the peripheral nerves, c) type X, A or Y zeolites which in addition to reducing sweat secretion also act as adsorbing agents for unpleasant odours, and d) botulinus toxin (toxin of the bacterium Chlostridium botulinum), and other substances which bring about a blocking of the release of the transmitter substance acetyl choline which is relevant for sweat secretion.

Odour absorbers are for example the phyllosilicates described in the laid-open patent specification DE-P 40 09 347, in particular montmorillonite, kaolinite, nontronite, saponite, hectorite, bentonite, smectite, and also zinc salts of ricinoleic acid for example. They also include deodorants, bactericidal or bacteriostatic deodorising substances, such as e.g. hexachlorophene, 2,4,4′-trichloro-2′-hydroxydiphenyl ether (Irgasan), 1,6-di-(4-chlorophenylbiguanido)hexane (chlorhexidine), 3,4,4′-trichlorocarbanilide, and the active agents described in the laid-open patent specifications DE-37 40 186, DE-39 38 140, DE-42 04 321, DE-42 29 707, DE-42 29 737, DE-42 37 081, DE-43 09 372, DE-43 24 219 and containing cation-active substances, such as e.g. quaternary ammonium salts and odour absorbers such as e.g. Grillocin® (combination of zinc ricinoleate and various additives) or triethyl citrate, optionally in combination with ion-exchange resins.

The amount of deodorising and/or antiperspirant active ingredients in the mixtures is preferably 0.01 to 20 wt. %, relative to the total weight of the preparations, particularly preferably 0.05 to 10 wt. %.

The mixture for use according to the invention can also in many cases advantageously be used in combination with preservatives. Preservatives chosen here are preferably those such as benzoic acid, esters and salts thereof, propionic acid and salts thereof, salicylic acid and salts thereof, 2,4-hexadienoic acid (sorbic acid) and salts thereof, formaldehyde and paraformaldehyde, 2-hydroxybiphenyl ether and salts thereof, 2-zinc sulfidopyridine-N-oxide, inorganic sulfites and bisulfites, sodium iodate, chlorobutanol, 4-ethyl mercury(II)-5-amino-1,3-bis(2-hydroxybenzoic acid, salts and esters thereof, dehydracetic acid, formic acid, 1,6-bis(4-amidino-2-bromophenoxy)-n-hexane and salts thereof, the sodium salt of ethyl mercury(II)-thiosalicylic acid, phenyl mercury and salts thereof, 10-undecenoic acid and salts thereof, 5-amino-1,3-bis(2-ethylhexyl)-5-methyl-hexahydropyrimidine, 5-bromo-5-nitro-1,3-dioxan, 2-bromo-2-nitro-1,3-propanediol, 2,4-dichlorobenzyl alcohol, N-(4-chlorophenyl)-N′-(3,4-dichlorophenyl)urea, 4-chloro-m-cresol, 2,4,4′-trichloro-2′-hydroxydiphenyl ether, 4-chloro-3,5-dimethyl phenol, 1,1′-methylene-bis(3-(1-hydroxymethyl-2,4-dioximidazolidin-5-yl)urea), poly(hexamethylene diguanide)hydrochloride, 2-phenoxyethanol, hexamethylene tetramine, 1-(3-chloroallyl)-3,5,7-triaza-1-azoniaadamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, benzyl alcohol, octopirox, 1,2-dibromo-2,4-dicyanobutane, 2,2′-methylene-bis(6-bromo-4-chlorophenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxypropan-2-ol, N-alkyl-(C₁₂-C₂₂)-trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxymethyl urea, 1,6-bis(4-amidinophenoxy)-n-hexane and salts thereof, glutaraldehyde, 5-ethyl-1-aza-3,7-dioxabicyclo(3.3.0)octane, 3-(4-chlorophenoxy)-1,2-propanediol, hyamine, alkyl-(C₈-C₁₈)-dimethylbenzyl ammonium chloride, alkyl-(C₈-C₁₈)-dimethylbenzyl ammonium bromide, alkyl-(C₈-C₁₈)-dimethylbenzyl ammonium saccharinate, benzyl hemiformal, 3-iodine-2-propinyl butyl carbamate, sodium hydroxymethylamino acetate or sodium hydroxymethylamino acetate.

Mixtures according to the invention, in particular dermatological mixtures, can also advantageously contain dyes and/or coloured pigments, particularly if they are intended for use in the area of decorative cosmetics. The dyes and coloured pigments can be selected from the corresponding positive list in the German cosmetics ordinance or the EU list of cosmetic colorants. In most cases they are identical to the dyes approved for foodstuffs. Advantageous coloured pigments are for example titanium dioxide, mica, iron oxides (e.g. Fe₂O₃ Fe₃O₄, FeO(OH)) and/or tin oxide. Advantageous dyes are for example carmine, Berlin blue, chromium oxide green, ultramarine blue and/or manganese violet. Mixtures of the cited active systems can also be used.

For use, topical mixtures (formulations) for use according to the invention are applied to the skin and/or hair in an adequate amount in the conventional way for cosmetics.

Other preferred embodiments of the invention can be seen from the following examples 1 to 10 and the appended claims.

### EXAMPLES 1-10

### Mixtures Comprising a Ceramide or Pseudoceramide and an Anti-Irritant for Strengthening the Barrier Function of the Skin

In the table below
1=skin-lightening day cream O/W
2=skin-soothing lotion with plant extracts O/W
3=after-sun balm
4=body spray
5=sunscreen lotion (O/W), broad-band protection
6=W/O night cream
7=shampoo
8=self-tanning cream
9=barrier repair cream O/W
10=roll-on antiperspirant/deodorant

| RAW MATERIAL NAME | | WEIGHT % | | | | | | | | | |
|---|---|---|---|---|---|---|---|---|---|---|---|
| (MANUFACTURER) | INCI | 1 | 2 | 3 | 4 | 5 | 6 | 7 | 8 | 9 | 10 |
| Pseudoceramides | | | | | | | | | | | |
| Pseudoceramide 176 | N-(1-dodecanoyl)-4- | | | 0.1 | 0.5 | | | | 0.1 | 0.1 | |
| | hydroxy-L-proline-(1- | | | | | | | | | | |
| | hexadecyl) ester | | | | | | | | | | |
| Pseudoceramide 391 | N-(1-hexadecanoyl)-4- | 0.5 | 1.0 | | | 0.1 | 1.0 | 0.2 | | 0.1 | 0.3 |
| | hydroxy-L-proline-(1- | | | | | | | | | | |
| | hexadecyl ester | | | | | | | | | | |
| Pseudoceramide 104 | 1,3-bis(N-(2- | | | | | 0.1 | | | | | |
| (PC104) (Pacific | hydroxyethyl) palmi- | | | | | | | | | | |
| Corporation) | toylamino)-2- | | | | | | | | | | |
| | hydroxypropane | | | | | | | | | | |
| Ceramides | | | | | | | | | | | |
| Ceramide 2 (Sederma) | Ceramide 2 | | | | | | | | | 0.2 | |
| Ceramide III | Ceramide 3 | 0.1 | | | | | | | | | |
| (Cosmoferm) | | | | | | | | | | | |
| Anti-irritants | | | | | | | | | | | |
| Allantoin (EMD | Allantoin | | 0.2 | | | | | | | | |
| Chemicals) | | | | | | | | | | | |
| -(-Alpha-)-Bisabolol, | Bisabolol | | | 0.1 | | | 0.1 | | | 0.1 | 0.1 |
| natural (Symrise) | | | | | | | | | | | |
| Dragosantol (Symrise) | Bisabolol | | | | 0.1 | 0.1 | | | | | |
| D-Panthenol (BASF) | Panthenol | | | 1.0 | | | | | | | |
| Ginger extract | | 1.0 | | | | | | | | | |
| Drago-Oat-Active | Water (aqua), butylene | | | | 1.0 | | | | | | |
| (Symrise) | gylcol, Avena sativa | | | | | | | | | | |
| | (oat) kernel extract | | | | | | | | | | |
| Extrapone Camomile GW | Glycerine, water (aqua), | | 0.5 | | | | | | | | |
| (Symrise) | Chamomilla recutita | | | | | | | | | | |
| | (matricaria) flower | | | | | | | | | | |
| | extract | | | | | | | | | | |
| Other ingredients | | | | | | | | | | | |
| Abil 350 (Degussa- | Dimethicone | 0.5 | 2.0 | 1.0 | | | | | 0.5 | 0.5 | |
| Goldschmidt) | | | | | | | | | | | |
| Allantoin (Merck) | Allantoin | | | 0.1 | | | | | | | |
| Aloe Vera Gel | Water (aqua), | | | 3.0 | | | 3.0 | | | | |
| Concentrate | Aloe barbadensis | | | | | | | | | | |
| 10/1 (Symrise) | leaf juice | | | | | | | | | | |
| Alugel 34 TH (Baer- | Aluminium stearate | | | | | | 1.0 | | | | |
| locher) | | | | | | | | | | | |
| Arbutin (Sabinsa) | β-Arbutin | 1.0 | | | | | | | | | |
| Sodium ascorbyl | Sodium ascorbyl phos- | 2.0 | | 1.0 | | | | | | | |
| phosphate | phate | | | | | | | | | | |
| (EMD Chemicals) | | | | | | | | | | | |
| Butylene glycol | Butylene glycol | | | 5.0 | | | | | | | |
| Carbopol ETD 2050 | Carbomer | | | | | 0.2 | | | | | |
| (Noveon) | | | | | | | | | | | |
| Carbopol Ultrez-10 | Carbomer | | 0.1 | | | | | | | | |
| (Noveon) | | | | | | | | | | | |
| Cetiol OE (Cognis) | Dicaprylyl ether | | | 4.0 | | | | | | | |
| Cetiol SB 45 (Cognis) | Butyrospermum Parkii | | | 1.0 | | | | | | | |
| | (shea butter) | | | | | | | | | | |
| Citric Acid 10% sol. | Citric acid | | | | | | | 0.3 | | | |
| Comperlan 100 (Cognis) | Cocamide MEA | | | | | | | 0.5 | | | |
| Dihydroxyacetone | Dihydroxyacetone | | | | | | | | 5.0 | | |
| (Merck) | | | | | | | | | | | |
| Dow Corning 246 Fluid | Cyclohexasiloxane and | | | | | 2.0 | | | | | |
| (Dow Corning) | cyclopentasiloxane | | | | | | | | | | |
| Dow Corning 345 Fluid | Cyclomethicone | | | | 0.5 | | | | | | |
| (Dow Corning) | | | | | | | | | | | |
| Dracorin CE (Symrise) | Glyceryl stearate | 5.0 | | | | | | | 5.0 | 1.5 | |
| | citrate | | | | | | | | | | |
| Dracorin GMS (Symrise) | Glyceryl stearate | | 2.0 | | | | | | | 2.0 | |
| Dracorin GOC (Symrise) | Glyceryl oleate | | | | 2.0 | | | | | | |
| | citrate, caprylic/ | | | | | | | | | | |
| | capric triglyceride | | | | | | | | | | |
| Drago-Beta-Glucan | Water (aqua), butylene | 0.3 | | | | | | | | | |
| (Symrise) | glycol, glycerine, | | | | | | | | | | |
| | Avena sativa | | | | | | | | | | |
| | (oat) kernel | | | | | | | | | | |
| | extract | | | | | | | | | | |
| Dragocid Liquid | Phenoxyethanol, | | 0.8 | 0.7 | | 0.7 | 0.8 | | | 0.8 | |
| (Symrise) | methylparaben, | | | | | | | | | | |
| | ethylparaben, | | | | | | | | | | |
| | butylparaben, | | | | | | | | | | |
| | propylparaben, | | | | | | | | | | |
| | isobutylparaben | | | | | | | | | | |
| Dragoderm (Symrise) | Glycerine, | | | | | | | 2.0 | | | |
| | Triticum vulgare | | | | | | | | | | |
| | (wheat) gluten, water | | | | | | | | | | |
| | (aqua) | | | | | | | | | | |
| Dragosan W/O Liquid | Polyglyceryl-3- | | | | | | 1.0 | | | | |
| (Symrise) | polyricinoleate, | | | | | | | | | | |
| | sorbitan isostearate | | | | | | | | | | |
| Dragosan W/O P | Sorbitan isostearate, | | | | | | 6.0 | | | | |
| (Symrise) | hydrogenated castor | | | | | | | | | | |
| | oil, ceresin, beeswax | | | | | | | | | | |
| | (Cera alba) | | | | | | | | | | |
| Dragoxat EH (Symrise) | Ethylhexyl ethyl- | 3.0 | 3.0 | | 4.0 | | | | 3.0 | | |
| | hexanoate | | | | | | | | | | |
| Dragoxat 89 (Symrise) | Ethylhexyl ethyl- | | | | | | | | | 2.0 | |
| | isononanoate | | | | | | | | | | |
| EDETA B powder (BASF) | Tetrasodium EDTA | | | | | | | 0.1 | | | |
| EDETA DB (BASF) | Disodium EDTA | | | | | 0.1 | | | 0.1 | | |
| Emulsiphos (Symrise) | Potassium cetyl phos- | | 2.0 | | | 1.5 | | | | 2.0 | |
| | phate, hydrogenated | | | | | | | | | | |
| | palm glycerides | | | | | | | | | | |
| ETD 2050 | Carbomer | | | | | | | | | | |
| Ethanol 96% | Ethanol | | | | | | | | 2.0 | | 30.0 |
| Eutanol G16 (Cognis) | Hexyldecanol | | | | | | | 1.0 | | 2.0 | |
| Extrapone Green Tea | Glycerine, water | | 0.2 | | | | | | | | |
| GW (Symrise) | (aqua), Camellia | | | | | | | | | | |
| | sinensis leaf | | | | | | | | | | |
| | extract | | | | | | | | | | |
| Extrapone Witch Hazel | Propylene glycol, | | | | | | 1.0 | | | | |
| Distillate colorless | Hamamelis virginiana | | | | | | | | | | |
| (Symrise) | (witch hazel) water, | | | | | | | | | | |
| | water (aqua), | | | | | | | | | | |
| | Hamamelis virginiana | | | | | | | | | | |
| | (witch hazel) | | | | | | | | | | |
| | extract | | | | | | | | | | |
| Extrapone Rosemary GW | Glycerine, water (aqua), | | 0.3 | | | | | | | 0.5 | |
| (Symrise) | Rosmarinus officinalis | | | | | | | | | | |
| | (rosemary) leaf extract | | | | | | | | | | |
| Farnesol (Symrise) | Farnesol | | | | | | | | | | 0.5 |
| Frescolat ML crist. | Menthyl lactate | | | 0.8 | | | | | | | |
| (Symrise) | | | | | | | | | | | |
| Genapol LRO liquid | Sodium laureth sulfate | | | | | | | 37.0 | | | |
| (Cognis) | | | | | | | | | | | |
| Givobio GZN (Seppic) | Zinc gluconate | | | | | | | | | 0.5 | |
| Glycerine 85% | Glycerine | 3.0 | 2.0 | 4.0 | | 4.7 | 2.0 | | 1.5 | 3.0 | |
| Hydrolite-5 (Symrise) | Pentylene glycol | | | | 5.0 | | | | 3.5 | | |
| Hydroviton (Symrise) | Water, glycerine, | | | | | | | | | 1.0 | |
| | sodium lactate, TEA | | | | | | | | | | |
| | lactate, serine, | | | | | | | | | | |
| | lactic acid, urea, | | | | | | | | | | |
| | sorbitol, sodium | | | | | | | | | | |
| | chloride, lauryl | | | | | | | | | | |
| | diethylenediamino- | | | | | | | | | | |
| | glycine, lauryl | | | | | | | | | | |
| | aminopropylglycine, | | | | | | | | | | |
| | allantoin | | | | | | | | | | |
| Irgasan DP 300 (Ciba | Triclosan | | | | | | | | | | 0.3 |
| Geigy) | | | | | | | | | | | |
| Isodragol (Symrise) | Triisononanoin | | 2.0 | | | | | | | 3.0 | |
| Isopropyl palmitate | Isopropyl palmitate | 4.0 | | | | | | | 4.0 | | |
| (Symrise) | | | | | | | | | | | |
| Karion F (Merck) | Sorbitol | | | | | | 2.0 | | | | |
| Keltrol RD (CP-Kelco) | Xanthan gum | 0.2 | 0.1 | | | | | | | | |
| Keltrol T | Xanthan gum | | | | | 0.2 | | | 0.3 | | |
| (Danby-Chemie) | | | | | | | | | | | |
| Kojic acid (Cosmeto- | Kojic acid | | | | | | | | | | |
| chem) | | | | | | | | | | | |
| Lanette 16 (Cognis) | Cetyl alcohol | 1.0 | | | | | | | 1.0 | | |
| Lanette O (Cognis) | Cetearyl alcohol | | 3.0 | | | 1.0 | | | | 2.0 | |
| Lara Care A-200 | Galactoarabinan | | | 0.3 | | | | | | | |
| (Rahn) | | | | | | | | | | | |
| Linoleic acid | Linoleic acid | | | | | | | | | 0.3 | |
| Magnesium chloride | Magnesium chloride | | | | | | 0.7 | | | | |
| (Merck) | | | | | | | | | | | |
| Merquat 550 (Ondeo | Polyquaternium-7 | | | | | | | 0.5 | | | |
| Nalco) | | | | | | | | | | | |
| NAOH 10% sol. | Sodium hydroxide | | | | | | | | | 0.3 | |
| Naringin (Exquim) | 4′,5,7- | | | | | | | 0.5 | 2.0 | | |
| | Trihydroxyflavone- | | | | | | | | | | |
| | 7-O-neohesperidoside | | | | | | | | | | |
| Sodium benzoate | Sodium benzoate | | | | | | | 0.5 | | | |
| Natrosol 250 HHR | Hydroxyethyl cellulose | | | | | | | | | | 0.3 |
| (Aqualon) | | | | | | | | | | | |
| Neo Heliopan 357 | Butyl methoxy | | | | | 1.0 | | | | | |
| (Symrise) | dibenzoylmethane | | | | | | | | | | |
| Neo Heliopan AP | Disodium phenyl dibenz- | | | | | 10 | | | | | |
| (Symrise) | imidazole tetrasulfonate | | | | | | | | | | |
| (10% as sodium salt) | | | | | | | | | | | |
| Neo Heliopan AV | Ethylhexyl methoxycin- | | | | | 3.0 | | | | | |
| (Symrise) | namate | | | | | | | | | | |
| Neo Heliopan Hydro | Phenylbenzimidazole | | | | | 6.7 | | | | | |
| (Symrise) | sulfonic acid | | | | | | | | | | |
| (15% as sodium salt) | | | | | | | | | | | |
| Neo Heliopan MBC | 4-Methylbenzylidene | | | | | 1.5 | | | | | |
| (Symrise) | camphor | | | | | | | | | | |
| Neo Heliopan OS | Ethylhexyl salicylate | | | | | 5.0 | | | | | |
| (Symrise) | | | | | | | | | | | |
| Neutral oil | Caprylic/capric | 6.0 | | | 4.0 | 2.0 | | | 6.0 | 10.0 | |
| | triglyceride | | | | | | | | | | |
| Oxynex 2004 (Merck) | BHT | | | | | | 0.1 | | | | |
| Palmitic acid | Palmitic acid | | | | | | | | | 0.3 | |
| Paraffin oil 5 | Paraffinum liquidum | | | | 4.0 | | | | | | |
| grade E (Parafluid) | | | | | | | | | | | |
| PCL Liquid 100 | Cetearyl ethylhexoate | 3.0 | 5.0 | | 7.0 | | | | | | |
| (Symrise) | | | | | | | | | | | |
| PCL Solid (Symrise) | Stearyl heptanoate, | | 2.0 | | | | | | | | |
| | stearyl caprylate | | | | | | | | | | |
| PCL-Liquid (Symrise) | Cetearyl ethylhexanoate, | | | | | | 12.0 | | 3.0 | | |
| | isopropyl myristate | | | | | | | | | | |
| Pemulen TR-2 (Noveon) | Acrylates/C10-30 alkyl | | | 0.3 | 0.2 | | | | | | |
| | acrylate crosspolymer | | | | | | | | | | |
| Propylene Glycol-1,2 | Propylene glycol | | 5.0 | | | | | | | | |
| 99P GC | | | | | | | | | | | |
| Retinyl palmitate in | Retinyl palmitate | | | | | | 0.2 | | | | |
| oil (DSM Nutritional | | | | | | | | | | | |
| Products) | | | | | | | | | | | |
| Sepigel 305 | Polyacrylamide, C13-14 | | | | | | | | 1.0 | | |
| | isoparaffin, laureth-7 | | | | | | | | | | |
| Sodium chloride | Sodium chloride | | | | | | | 1.0 | | | |
| Sodium hydroxide | Sodium hydroxide | | 0.3 | 0.6 | 0.4 | | | | | | |
| (10% sol.) | | | | | | | | | | | |
| Solubilizer 611674 | PEG-40 hydrogenated | | | | | | | | | | 2.0 |
| (Symrise) | castor oil, trideceth-9, | | | | | | | | | | |
| | water (aqua) | | | | | | | | | | |
| Sunflower oil | Helianthus annuus (sun- | | | | | | 5.0 | | | | |
| (Wagner) | flower) seed oil | | | | | | | | | | |
| Sweet almond oil | Prunus dulcis | | | | | | 5.0 | | | | |
| (Wagner) | | | | | | | | | | | |
| Symatrix (Symrise) | Maltodextrin, Rubus | | 0.1 | | | 0.3 | 1.0 | | | | |
| | fruticosus (blackberry) | | | | | | | | | | |
| | leaf extract, sodium | | | | | | | | | | |
| | ascorbyl phosphate | | | | | | | | | | |
| Symdiol 68 (Symrise) | 1,2-Hexanediol, | 0.5 | | | | | | | | | |
| | caprylylglycol | | | | | | | | | | |
| Symrise Fragrance | Fragrance | 0.3 | 0.3 | 0.3 | 0.2 | 0.4 | 0.4 | 0.5 | 0.3 | 0.3 | 1.0 |
| Tamasterol (Tama | Phytosterols | | | | | | | | | 0.3 | |
| Biochemicals) | | | | | | | | | | | |
| Tego Betaine L7 | Cocamidopropyl betaine | | | | | | | 6.0 | | | |
| (Degussa) | | | | | | | | | | | |
| Tegosoft PC 31 | | | | | | | | | | 0.3 | |
| (Degussa) | | | | | | | | | | | |
| Tegosoft TN (Degussa) | C12-15 Alkyl benzoate | | | 5.0 | | 5.0 | | | | | |
| Triethanolamine, 99% | Triethanolamine | | | | | 0.5 | | | | | |
| Tocopherol acetate | Tocopheryl acetate | | | 0.5 | | 0.5 | 3.0 | | | 0.3 | |
| (DSM Nutritional | | | | | | | | | | | |
| Products) | | | | | | | | | | | |
| Zirkonal L 450 | Aluminium zirconium | | | | | | | | | | 37.0 |
| (BK Giulini) | pentachlorohydrate | | | | | | | | | | |
| | (40% aqueous solution) | | | | | | | | | | |
| Water, demineralized | Water (aqua) | to 100 | to 100 | to 100 | to 100 | to 100 | to 100 | to 100 | to 100 | to 100 | to 100 |

1. A process for strengthening the barrier function of undamaged skin in particular against allergens and/or for preventing or inhibiting an allergic reaction of undamaged skin on contact with an allergenic active ingredient, comprising:
applying to undamaged skin an effective amount of a mixture comprising (a) a ceramide and/or a pseudoceramide and (b) an anti-irritant.

2. A process for the cosmetic treatment of undamaged skin with a cosmetic active ingredient having an allergenic side-effect, comprising:
applying to undamaged skin an effective amount of a mixture comprising (a) a ceramide and/or a pseudoceramide and (b) an anti-irritant and optionally (c) an acceptable support to enhance skin barrier functions, and
applying a cosmetic active ingredient having an allergenic side-effect to skin treated with said mixture.

3. (canceled)

4. A process according to claim 1, wherein the anti-irritant is selected from the group consisting of bisabolol, panthenol, ginger extracts and mixtures thereof.

5. A process according to claim 1, wherein said pseudoceramide is selected from the group consisting of 1,3-bis-(N-(2-hydroxyethyl)alkylamino)-2-hydroxypropanes, N-(2-hydroxyethyl)-3-oxo-2-alkyl alkylamides, N-acyl hydroxyamino acid esters and mixtures thereof.

6. A process according to claim 5, wherein said pseudoceramide is selected from the group consisting of 1,3-bis-(N-(2-hydroxyethyl)palmitoylamino)-2-hydroxypropane, N-(2-hydroxyethyl)-3-oxo-2-tetradecyl octadecanamide, N-acyl hydroxyamino acid esters and mixtures thereof.

7. A process according to claim 1, wherein said pseudoceramide is an N-acyl hydroxyamino acid ester and the anti-irritant is bisabolol.

8. A process according to claim 5, wherein said pseudoceramide comprises an N-acyl hydroxyamino acid ester of formula I, 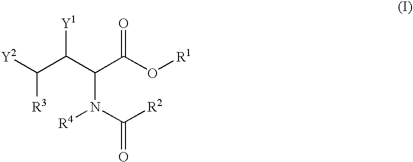
wherein
R¹ is a linear, branched or cyclic alkyl or alkenyl group having 5 to 50 carbon atoms, which is optionally substituted with one or more hydroxyl radicals,
R² is a linear or branched alkyl or alkenyl group having 1 to 49 carbon atoms, which is optionally substituted with one or more hydroxyl radicals,
Y¹ and Y² are mutually independently hydrogen or hydroxyl,
R³ and R⁴ either mutually independently stand for hydrogen or linear or branched alkyl or alkenyl groups having 1 to 10 carbon atoms or
R³ and R⁴ together represent an alkylene radical having 1 to 3 carbon atoms and together with the chain between R³ and R⁴ form a 5-, 6- or 7-membered heterocyclic ring, wherein for its part this alkylene radical is optionally substituted by 1 to 3 linear or branched alkyl or alkenyl groups or by 1 to 3 hydroxyl radicals.

9. A process according to claim 8, wherein
R¹ is a linear, branched or cyclic alkyl or alkenyl group having 5 to 24 carbon atoms, which is optionally substituted with 1 to 6 hydroxyl radicals.

10. A process according to claim 8, wherein
R² is a linear or branched alkyl or alkenyl group having 2 to 23 carbon atoms, which is optionally substituted with 1 to 6 hydroxyl radicals.

11. A process according to claim 10, wherein
R² is a linear or branched alkyl or alkenyl group having 2 to 23 carbon atoms, which is optionally substituted with 1 to 3 hydroxyl radicals.

12. A process according to claim 8, wherein one of the two groups Y¹ and Y² denotes a hydroxyl radical and the other denotes a hydrogen atom.

13. A process according to claim 8, wherein
R³ and R⁴ mutually independently stand for hydrogen or linear or branched alkyl or alkenyl groups having 1, 2, 3 or 4 carbon atoms or
R³ and R⁴ together stand for the alkylene radicals —CH₂—, —CH₂—CH₂—, —CH(OH)—, —CH(OH)—CH₂— or —CH₂—CH(OH)—.

14. A process according to claim 8, wherein
R³ and R⁴ are hydrogen atoms and at the same time Y¹ and Y² are mutually independently hydrogen atoms or hydroxyl radicals, or
R³ and R⁴ together represent a —CH₂— or a —CH(OH)— group and together with the chain between R³ and R⁴ form a 5-membered heterocyclic ring and at the same time Y¹ and Y² are hydrogen atoms or hydroxyl radicals.

15. A process according to claim 14, wherein
R³ and R⁴ are hydrogen atoms and at the same time Y¹ represents a hydroxyl radical and Y² a hydrogen atom (N-acyl threonine alkyl ester) or
R³ and R⁴ together represent a —CH₂— group and together with the chain between R³ and R⁴ form a 5-membered heterocyclic ring and one of the two radicals Y¹ and Y² represents a hydroxyl radical (N-acyl hydroxyproline ester).

16. A process according to claim 15, wherein
R¹ denotes an unbranched alkyl or alkenyl radical having 5 to 24 carbon atoms and R² denotes an unbranched alkyl or alkenyl radical having 2 to 23 carbon atoms.
